(19) United States
(12) Patent Application Publication
(10) Pub. No.: US 2004/0006323 A1
(43) Pub. Date: Jan. 8, 2004

(54) Garments using elastic strands to enhance performance of elastic barrier adhessive

(76) Inventors: Gregory K. Hall, Menasha, WI (US); Tim J. Janssen, Hortonville, WI (US); Michael J. Garvey, Appleton, WI (US); Thomas W. Odorzynski, Green Bay, WI (US)

Correspondence Address: PAULEY PETERSEN KINNE & ERICKSON, 2800 WEST HIGGINS ROAD, SUITE 365, HOFFMAN ESTATES, IL, 60195, US

(21) Appl. No.: 10/187,682

(22) Filed: Jul. 2, 2002

(51) Int. Cl. A61F 13/15; A61F 13/20

(52) U.S. Cl. 604/385.24

(57) ABSTRACT

Garments have elastomeric composites and elastomeric composite laminates including reinforcement strands incorporated into an elastomeric adhesive film. The strands may vary in terms of levels of tension. Facing layers, such as nonwoven webs, can be laminated to both surfaces of the elastomeric composite to form laminates. The facing layer laminates can then be incorporated to provide expandable areas such as elastomeric cuff areas or containment flaps for garments to improve the elastic characteristics of such areas thereby providing good aesthetics and performance for such garments. Alternatively, the elastomeric composite of reinforcement strands incorporated into an elastomeric adhesive film can be utilized directly between two primary layers of a garment, e.g., the liner and the outer cover, to produce a laminate which is an integral part of the garment.

### BACKGROUND OF THE INVENTION

This invention is directed to absorbent articles with elastic composite materials including elastomeric adhesive films reinforced with elastic strands which significantly improve decay and overall elastic properties of the material thereby producing a better garment fit.

It is desired that absorbent articles and garments, and especially garments such as diapers, training pants or incontinence garments, without limitation referred to generically sometimes for ease of explanation as “diapers”, provide a close, comfortable fit about the body of the wearer and contain body exudates while maintaining skin health. In certain circumstances, it is also desirable that such garments are capable of being pulled up or down over the hips of the wearer to allow the wearer or care giver to easily pull the article on and easily remove the article. Other garment openings such as sleeve or pant cuffs and necklines may benefit from similar elasticizing.

Personal care products including such diapers and sanitary pads often are made with a top sheet material (also referred to as a cover sheet or liner), an absorbent core which is the primary liquid retention layer, and a liquid impervious back sheet, or outer layer. Some such items may also have a surge layer for fluid uptake and distribution, or other specialized layers between the top sheet and absorbent core, and additional gasketing, or containment, flaps within the product. Absorption and retention of fluid, comfort, and avoidance of leakage are the functions desired of such products. Thus, garments often include elasticized portions to create a gasket-like fit around certain openings, such as waist openings and leg openings. Laminates made from conventional elastic strands and elastic attachment adhesive are often used to create such elasticized portions. However, such laminates can be rough and uncomfortable. Furthermore, such laminates may cause red-marking on a wearer's skin if the fit is too tight and may result in leakage from the garment if the fit is too loose.

Elastic barrier adhesive films are currently recognized as suitable for use in the manufacture of personal care articles. More particularly, elastic barrier adhesive films can be used to bond facing materials, such as spunbond, to one another while simultaneously elasticizing the resulting laminate. The resulting laminate can be used to form an elastomeric portion of an absorbent article, such as a region surrounding a waist opening and/or a leg opening.

However, current elastic barrier adhesive films, when used in combination with spunbond layers to form spunbond laminates, may display noticeable tension decay. Additional tackifiers, or adhesives, can be added to the laminates to improve adhesion, but when tackifiers set they become rigid and therefore negatively affect the softness and pliability of the laminates, thereby leading to a loss of performance or aesthetics, or both, for the garment.

There has been a desire in the art to make incontinence garments, such as diapers, better fitting. One technique for rendering a better fit is to have at least some of the functional layers, e.g., the top and back sheets or the containment flaps, expandable, especially laterally or transversely, in the waist area of the garment. It is known in the art that expandability of the garment can be limited by the least expandable layer when said layers are connected in the constructed garment.

There is a further need or desire for a garment utilizing elastomeric laminates so as to create elasticized portions of the garment, wherein the laminate does not display high tension decay. There is a further need or desire for an elastomeric laminate that possesses a soft feel and comfortable fit, while providing sufficient tension to minimize leakage resulting in a garment of improved performance or aesthetics, or both.

### SUMMARY OF THE INVENTION

In response to the discussed difficulties and problems encountered in the prior art, garments utilizing new elastomeric composites have been discovered.

In certain aspects of the present invention, any garment opening such as a waist opening, sleeve or leg cuffs, or necklines may benefit from elasticizing. The margins of any garment opening may hereinafter be collectively referred to as “cuffs” or “cuff areas”. Certain aspects of the present invention may provide any one of an elasticized cuff area, non-cuff area, or a containment flap, having extensibility and elasticity for improved fit and the reduced leakage of exudates.

The present invention is directed to garments utilizing elastic composites, and laminates incorporating such elastic composites, to provide superior elastic properties without delamination of the composites. The elastic composites utilized in certain aspects of the invention are made up of a combination of reinforcing strands and elastomeric adhesive film. A layer of spunbond or other facing material can be laminated along one, or both, surfaces of the film to provide the elastic composite laminates of the invention. Alternatively, it is envisioned that laminates according to the present invention may be produced utilizing the stranded elastic barrier adhesive film composite placed between primary garment layers such as the back sheet, or outer cover, and liner of the garment. The combination of reinforcing strands and the elastomeric adhesive film significantly improves the rate and extent of tension compared to spunbond laminates including elastomeric adhesive film without reinforcing strands. Optionally, the reinforcing strands may enable the composite tension to be tunable while preserving the soft feel and aesthetic properties of the laminate.

A method of making these elastic composites and elastic composite laminates is further discussed herein. This method includes the steps necessary for incorporating the elastic strands into an elastomeric adhesive film. The elastic strands can be unrolled from one or more rolls while the elastomeric adhesive film is extruded onto a chill roll and the two or more materials joined at a compressing nip.

If desired, the tension in the elastic strands can be adjusted by the manner in which the strands are stretched. For example, to create greater tension, the strands can be stretched a greater extent when being incorporated with the elastomeric adhesive film. Another way to adjust tension is to adjust the add-on rate of the strands. Alternatively, or in addition to these methods of adjusting tension, the strands may very in thickness and/or may vary in composition.

With the foregoing in mind, it is a feature and advantage of the invention to provide garments utilizing elastic composites and elastic composite laminates having improved tension properties. Certain aspects of the invention also contemplate methods of making garments including such elastic composites and elastic composite laminates.

### BRIEF DESCRIPTION OF THE DRAWINGS

The accompanying drawings are presented as an aid to explanation and understanding of various aspects of the present invention only and are not to be taken as limiting the present invention. The drawings are not necessarily to scale, nor should they be taken as photographically accurate depictions of real objects unless otherwise stated.

FIG. 1 illustrates a first garment according to the present invention, in this case an exemplary diaper.

FIG. 2 is a plan view of an elastic composite suitable for use with the invention.

FIG. 3 is a plan view of another embodiment of an elastic composite suitable for use with the invention.

FIG. 4 is a cross-sectional view, taken along line 4-4 of FIG. 2, of another embodiment of an elastic composite suitable for use with the invention.

FIG. 5 is a perspective view of an elastic composite laminate suitable for use with the invention.

FIG. 6 is a cross-sectional view, taken along line 6-6 of FIG. 5, of another embodiment of an elastic composite laminate suitable for use with the invention.

FIG. 7 illustrates a representative process for making the elastic composites and elastic composite laminates suitable for use with the invention.

FIG. 8 is a perspective view of a garment having an elastic composite laminate around the leg openings and waist opening.

FIG. 9 is a graph of percentage load loss versus time comparing samples of laminate with and without added spandex reinforcing fibers.

FIGS. 10-13 are graphs of load versus percentage elongation for comparison of samples of laminate with and without added spandex reinforcing fibers.

### DEFINITIONS

Within the context of this specification, each term or phrase below will include the following meaning or meanings.

“Bonded” refers to the joining, adhering, connecting, attaching, or the like, of at least two elements. Two elements will be considered to be bonded together when they are bonded directly to one another or indirectly to one another, such as when each is directly bonded to intermediate elements.

“Decitex” or “dTex” refers to a measure of the linear density of fibers in grams per 1000 meters of fiber.

“Elastic tension” refers to the amount of force per unit width required to stretch an elastic material (or a selected zone thereof) to a given percent elongation.

“Elastomeric” and “elastic” are used interchangeably to refer to a material or composite that is generally capable of recovering its shape after deformation when the deforming force is removed.

“Elongation”, refers to the capability of an elastic material to be stretched a certain distance, such that greater elongation refers to a material capable of being stretched a greater distance than a material having lower elongation. “Stretchability” and “expandability” will generally be considered as having the same meaning.

“Film” refers to a thermoplastic film made using a film extrusion process, such as a cast film or blown film extrusion process. The term includes apertured films, slit films, and other porous films which constitute liquid transfer films, as well as films which do not transfer liquid.

“Garment” includes personal care garments, medical garments, and the like. The term “disposable garment” includes garments which are typically disposed of after 1-5 uses. The term “personal care garment” includes diapers, training pants, swim wear, absorbent underpants, adult incontinence products, feminine hygiene products, and the like. The term “medical garment” includes medical (i.e., protective and/or surgical) gowns, caps, gloves, drapes, face masks, and the like. The term “industrial workwear garment” includes laboratory coats, cover-alls, and the like.

“Incorporate” refers to the process of combining two or more elements into a single structure intended to be inseparable.

“Layer” when used in the singular can have the dual meaning of a single element or a plurality of elements.

As used herein, the term “machine direction” means the length of a fabric in the direction in which it is produced. The term “cross direction” or “cross machine direction” means the width of fabric, i.e. a direction generally perpendicular to the machine direction.

“Meltblown fiber” refers to fibers formed by extruding a molten thermoplastic material through a plurality of fine, usually circular, die capillaries as molten threads or filaments into converging high velocity gas (e.g., air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed for example, in U.S. Pat. No. 3,849,241 to Butin et al. Meltblown fibers are microfibers which may be continuous or discontinuous, are generally smaller than about 0.6 denier, and are generally self bonding when deposited onto a collecting surface.

“Nonwoven” and “nonwoven web” refer to materials and webs of material having a structure of individual fibers or filaments which are interlaid, but not in an identifiable manner as in a knitted fabric. The terms “fiber” and “filament” are used herein interchangeably. Nonwoven fabrics or webs have been formed from many processes such as, for example, meltblowing processes, spunbonding processes, air laying processes, and bonded carded web processes. The basis weight of nonwoven fabrics is usually expressed in ounces of material per square yard (osy) or grams per square meter (gsm) and the fiber diameters are usually expressed in microns. (Note that to convert from osy to gsm, multiply osy by 33.91.)

“Polymers” include, but are not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configurations of the material. These configurations include, but are not limited to isotactic, syndiotactic and atactic symmetries.

“Spandex” refers to elastic fibers of a long chain synthetic polymer comprised of at least 85% of a segmented polyurethane. “Spunbond fiber” refers to small diameter fibers which are formed by extruding molten thermoplastic material as filaments from a plurality of fine capillaries of a spinnerette having a circular or other configuration, with the diameter of the extruded filaments then being rapidly reduced as taught, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Petersen, and U.S. Pat. No. 3,542,615 to Dobo et al., each of which is incorporated herein in its entirety by reference. Spunbond fibers are quenched and generally not tacky when they are deposited onto a collecting surface. Spunbond fibers are generally continuous and often have average deniers larger than about 0.3, more particularly, between about 0.6 and 10.

“Strand” refers to an article of manufacture whose width is less than a film and is suitable for incorporating into a film, according to the present invention.

“Thermoplastic” describes a material that softens when exposed to heat and which substantially returns to a nonsoftened condition when cooled to room temperature.

“Thermoset” describes a material that is capable of becoming permanently cross-linked.

“Vertical filament stretch-bonded laminate” or “VF SBL” refers to a stretch-bonded laminate made using a continuous vertical filament process, as described herein.

These terms may be defined with additional language in the remaining portions of the specification.

### DETAILED DESCRIPTION

The various aspects and embodiments of the invention will be described in the context of disposable absorbent articles, and more particularly referred to, without limitation and by way of illustration only, as a disposable diaper. It is, however, readily apparent that the present invention could also be employed to produce other products or garments, such as feminine care articles, various incontinence garments, medical garments and any other disposable garments. Typically, the disposable garments are intended for limited use and are not intended to be laundered or otherwise cleaned for reuse. A disposable diaper, for example, is discarded after it has become soiled by the wearer.

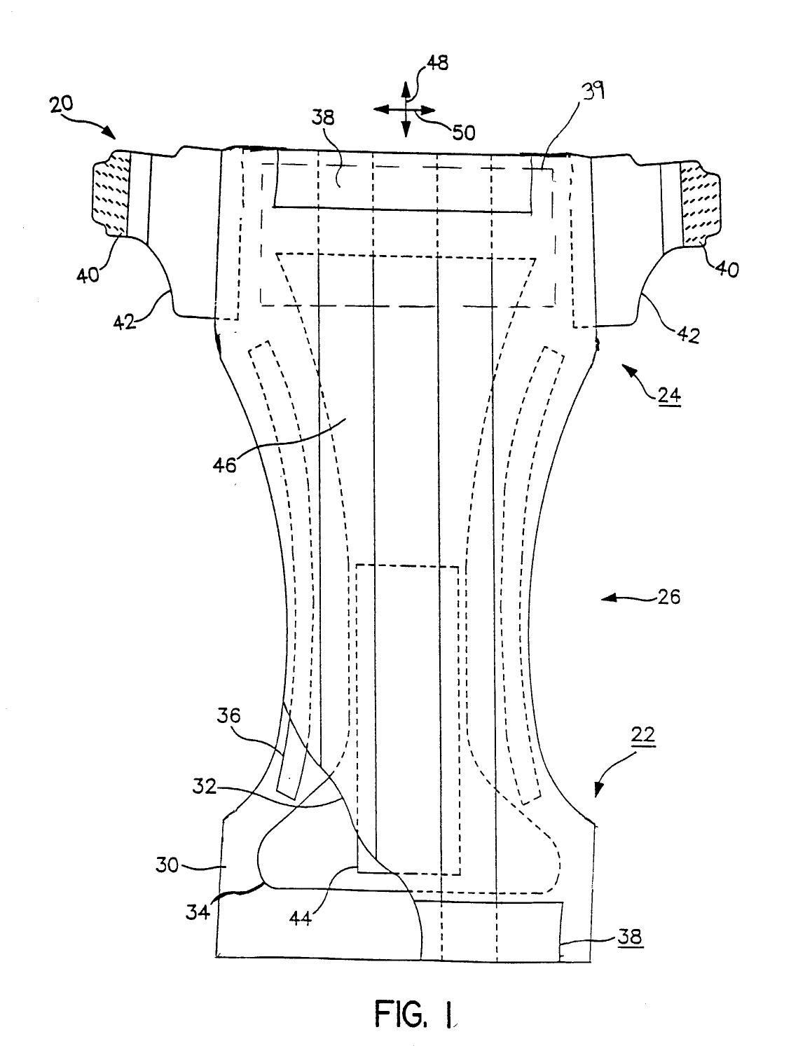

FIG. 1 is a representative plan view of an absorbent article, such as disposable diaper 20, in its flat-out, or unfolded state. Portions of the structure are partially cut away to more clearly show the interior construction of diaper 20. The surface of the diaper 20 which contacts the wearer is facing the viewer.

With reference to FIG. 1, the disposable diaper 20 generally defines a front waist section 22, a rear waist section 24, and an intermediate section 26 which interconnects the front and rear waist sections. The front and rear waist sections 22 and 24 include the general portions of the diaper which are constructed to extend substantially over the wearer's front and rear abdominal regions, respectively, during use. The intermediate section 26 of the diaper includes the general portion of the diaper that is constructed to extend through the wearer's crotch region between the legs. Thus, the intermediate section 26 is an area where repeated liquid surges typically occur in the diaper.

The diaper 20 includes, without limitation, an outer cover, or back sheet 30, a liquid permeable bodyside liner, or topsheet, 32 positioned in facing relation with the back sheet 30, and an absorbent core, or body, being the primary liquid retention structure, 34, such as an absorbent pad, which is located between the back sheet 30 and the topsheet 32. The back sheet 30 defines a length, or longitudinal direction 48, and a width, or lateral direction 50 which, in the illustrated embodiment, coincide with the length and width of the diaper 20. The liquid retention structure 34 generally has a length and width that are less than the length and width of the back sheet 30, respectively. Thus, marginal portions of the diaper 20, such as marginal sections of the back sheet 30, may extend past the terminal edges of the liquid retention structure 34. In the illustrated embodiment, for example, the back sheet 30 extends outwardly beyond the terminal marginal edges of the liquid retention structure 34 to form side margins and end margins of the diaper 20. The topsheet 32 is generally coextensive with the back sheet 30 but may optionally cover an area which is larger or smaller than the area of the back sheet 30, as desired.

The diaper 20 may include leg elastics 36 which are constructed to operably tension the side margins of the diaper 20 to provide elasticized leg bands which can closely fit around the legs of the wearer to reduce leakage and provide improved comfort and appearance. Waist elastics 38 are employed to elasticize the end margins of the diaper 20 to provide elasticized waistbands. The waist elastics 38 are configured to provide a resilient, comfortably close fit around the waist of the wearer. A zone of expansion 39 is indicated for the diaper 20. The zone of expansion 39 is that area of the diaper 20 most likely to be made laterally expandable to increase fit and comfort of some embodiments of the diaper 20. The person having ordinary skill in the art will appreciate that other areas, such as the front waist section 22, or the entire area of the diaper 20 such as covered by top sheet 32, may be made expandable. Any expandable areas of the diaper 20 may utilize the elastic composites set forth herein for increased functionality and aesthetics.

In the illustrated embodiment, the diaper 20 includes a pair of side panels 42 to which fasteners 40, indicated as the hook portion of a hook and loop fastener, are attached. Generally, the side panels 42 are attached to the side edges of the diaper 20 in one of the waist sections 22, 24 and extend laterally outward therefrom. The side panels 42 may be expandable. For example, the side panels 42, or indeed, any precursor component webs of the garment, may be a laminate set forth herein and may utilize an elastomeric facing material such as a neck-bonded laminate (NBL) or stretch-bonded laminate (SBL) material. Methods of making such materials are well known to those skilled in the art and are described in U.S. Pat. No. 4,663,220 issued May 5, 1987 to Wisneski et al., U.S. Pat. No. 5,226,992 issued Jul. 13, 1993 to Morman, and European Patent Application No. EP 0 217 032 published on Apr. 8, 1987 in the names of Taylor et al. Examples of absorbent articles that include elasticized side panels and selectively configured fastener tabs are described in PCT Patent Application No. WO 95/16425 published Jun. 22, 1995 to Roessler; U.S. Pat. No. 5,399,219 issued Mar. 21, 1995 to Roessler et al.; U.S. Pat. No. 5,540,796 to Fries; U.S. Pat. No. 5,595,618 to Fries and U.S. Pat. No. 5,496,298 to Kuepper et al.

The diaper 20 may also include a surge management layer 44, located between the topsheet 32 and the liquid retention structure 34, to rapidly except fluid exudates and distribute the fluid exudates to the liquid retention structure 34 within the diaper 20. The diaper 20 may further include a ventilation layer (not illustrated) located between the liquid retention structure 34 and the back sheet 30 to insulate the back sheet 30 from the liquid retention structure 34 to reduce the dampness of the garment at the exterior surface of the back sheet 30. Examples of suitable surge management layers 44 are described in U.S. Pat. No. 5,486,166 to Bishop; U.S. Pat. No. 5,490,846 to Ellis; U.S. Pat. No. 5,364,382 to Latimer et al.; and U.S. Pat. No. 5,429,629 to Latimer et. al.

As representatively illustrated in FIG. 1, the disposable diaper 20 may also include a pair of expandable containment flaps 46 which are configured to provide a barrier to the lateral flow of body exudates. The containment flaps 46 may be located along the laterally opposed side edges of the diaper 20 adjacent the side edges of the liquid retention structure 34. Each containment flap 46 typically defines an unattached edge which is configured to maintain an upright, perpendicular configuration in at least the intermediate section 26 of the diaper 20 to form a seal against the wearer's body.

The present invention incorporates elastic composites and elastic composite laminates having superior elastic and adhesion properties. The composites and laminates can be incorporated into any suitable article, such as personal care garments, medical garments, and industrial workwear garments. More particularly, the elastic composites and elastic composite laminates are suitable for use in diapers, training pants, swim wear, absorbent underpants, adult incontinence products, feminine hygiene products, protective medical gowns, surgical medical gowns, caps, gloves, drapes, face masks, laboratory coats, and coveralls.

A number of elastomeric components are known for use in the design and manufacture of such articles. For example, disposable absorbent articles are known to contain expandable and elasticized leg cuffs, elasticized waist portions including cuff areas thereof, elasticized containment flaps, elasticized side panels and fastening tabs. The elastic composites and laminates of this invention may be applied to any suitable article to form such expandable and elasticized areas.

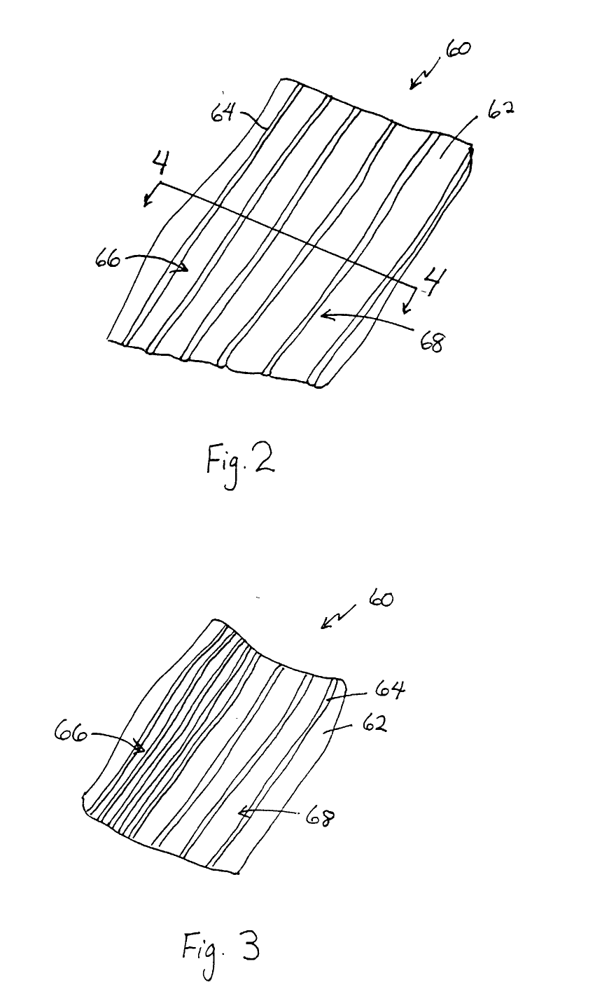

As shown in FIG. 2, an elastomeric composite 60 of the invention includes an elastomeric adhesive film 62 with a number of elastic reinforcing strands 64 incorporated therein. A first region 66 of the composite 60 may have a different amount of tension than a second region 68 of the composite 60. For example, the elastic strands 64 in the first region 66 may impart greater tension than the elastic strands 64 in the second region 68. Furthermore, the composite 60 may include multiple regions of differing tension.

Tension within the regions 66, 68 may be controlled through percentage stretch of the strands 64 during incorporation into the elastomeric adhesive film 62, and/or through the amount of strand add-on or thickness, with greater stretch and greater add-on or thickness each resulting in higher tension. Tension can also be controlled by varying strand geometries, spacing between strands, and/or varying the composition of the strands. It will be appreciated that the strands 64 may be laid out periodically, non-periodically, and in various spacings, groupings, sizes, and compositions of elastic material according to the effect desired from the composite 60 and the use to which it is put.

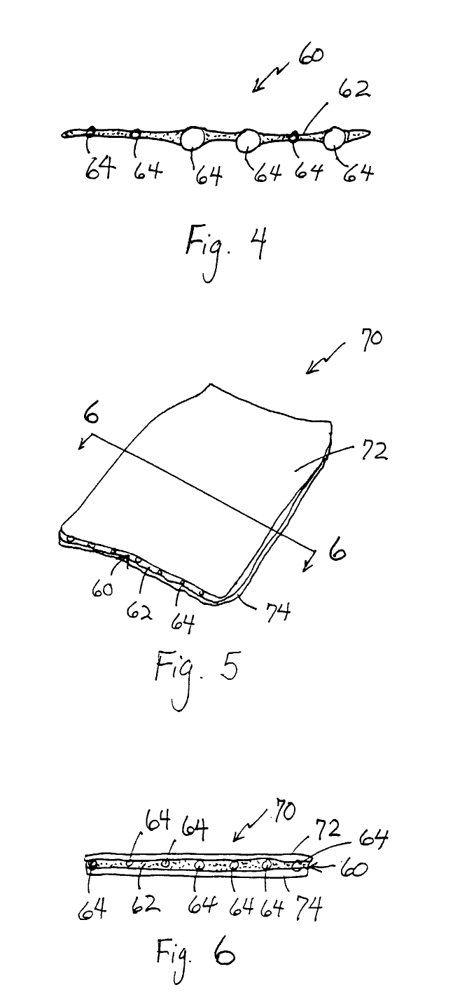

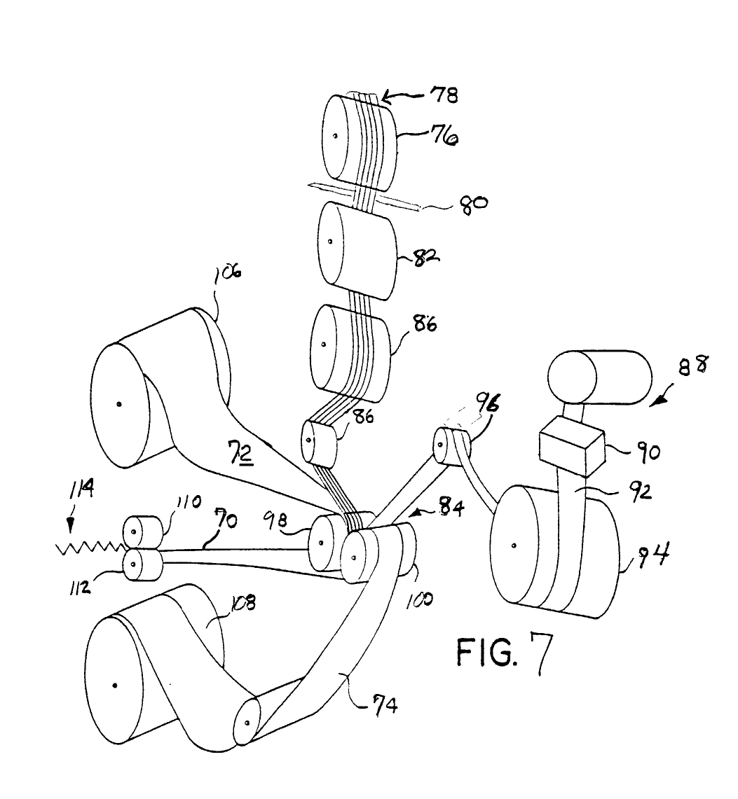

As shown in FIG. 3, for example, a group of strands 64 in one region 66 of the composite 60 can be spaced apart much more closely than another group of strands 64, resulting in greater tension in the region in which the strands 64 are more closely spaced. As another example, FIG. 4 illustrates a cross-sectional view of the composite 60 having unequally sized elastic strands 64 with some strands having a larger diameter, and thus higher tension, than others. While referred to as being of different diameter, it will be appreciated that the strands 64 need not be circular in cross-section within the context of this invention. Furthermore, the strands 64 of different size or composition may be intermingled within groupings in regular or irregular patterns.

The elastomeric adhesive film 62 is suitably made up of an elastomeric, hot melt, pressure-sensitive adhesive having an adhesive bond strength, as determined by the test method set forth below, of at least 100 grams force per inch (2.54 cm) width, suitably of at least 200 grams force per inch (2.54 cm) width, alternatively of at least 400 grams force per inch (2.54 cm) width, alternatively of at least from about 200 grams force per inch (2.54 cm) width to about 700 grams force per inch width. The elastomeric, hot melt, pressure-sensitive adhesive may be applied to a chill roll or similar device, in the form of a strand or ribbon. The strand or ribbon is then stretched and thinned to form the film 62. The film suitably has a thickness of about 0.001 inch (0.025 mm) to about 0.05 inch (1.27 mm), alternatively of from about 0.001 inch (0.025 mm) to about 0.01 inch (0.25 mm), and a width of from about 0.05 inch (1.27 mm) to about 3.0 inches (7.62 cm), alternatively of from about 0.5 inch (1.27 cm) to about 1.5 inches (3.81 cm). The elastomeric, adhesive film 62 may also be capable of imparting barrier properties in an application.

Suitable elastomeric, hot melt, pressure-sensitive adhesives from which the elastomeric adhesive film 62 may be made include elastomeric polymers, tackifying resins, plasticizers, oils and antioxidants. Such elastomeric, hot melt, pressure-sensitive adhesives are available from Bostik Findley of Middleton, Mass., under the trade designations H2503, H2504, HX2567-02, and HX2695-F01 as used with the examples set forth below. Other formulations or types of elastic barrier adhesive may also be suitable for use within certain aspects of the present invention.

The elastomeric adhesive film 62 suitably may have an elongation of at least 50 percent, alternatively of at least 150 percent, alternatively of from about 50 percent to about 200 percent. The elastomeric adhesive film 62 may further suitably have a retractive force of less than about 400 grams force per inch (2.54 cm) width, alternatively of less than about 275 grams force per inch (2.54 cm) width, alternatively of from about 100 grams force per inch (2.54 cm) width to about 250 grams force per inch (2.54 cm) width, as determined by a tensile tester at one minute of stretching the film to a ninety percent elongation.

The elastomeric adhesive film 62 is capable not only of introducing a degree of elasticity to facing materials but is also capable of providing a construction adhesive function. That is, the film 62 adheres together the facing materials or other components with which it is in contact. It is also possible that the film does not constrict upon cooling but, instead, tends to retract to approximately its original dimension after being elongated during use in a product.

Materials suitable for use in preparing the elastic reinforcing strands 64 include diblock, triblock, tetrablock, or other multi-block elastomeric copolymers such as olefinic copolymers, including styrene-isoprene-styrene, styrene-butadiene-styrene, styrene-ethylene/butylenes-styrene, or styrene-ethylene/propylene-styrene, which may be obtained from Kraton Polymers, Inc., under the trade designation KRATON® elastomeric resin; polyurethanes, including spandex materials such as those available from E. I. Du Pont de Nemours Co., under the trade name LYCRA® polyurethane, or GLOSPAN® from Globe Manufacturing Company; polyamides, including polyether block amides available from AtoFina chemical company under the trade name PEBAX® polyether block amide; polyesters, such as those available from E. I. Du Pont de Nemours Co., under the trade name HYTREL® polyester; and single-site or metallocene-catalyzed polyolefins having density less than about 0.89 grams/cubic centimeter, available from Dow Chemical Co. under the trade name AFFINITY®.

Alternatively, the elastic strands 64 can be made of a polymer that is not thermally processable, such as the aforementioned LYCRA® or GLOSPAN® spandex, or cross-linked natural rubber in film or fiber form. Thermoset polymers and polymers such as spandex, unlike the thermoplastic polymers, once cross-linked cannot be thermally processed, but can be obtained on a spool or other form and can be stretched and applied to the strands in the same manner as thermoplastic polymers. As another alternative, the elastic strands 64 can be made of a thermoset polymer, such as AFFINITY®, available from Dow Chemical Co., that can be processed like a thermoplastic, i.e. stretched and applied, and then treated with radiation, such as electron beam radiation, gamma radiation, or UV radiation to cross-link the polymer, or use polymers that have functionality built into them such that they can be moisture-cured to cross-link the polymer, thus resulting in a polymer and the enhanced mechanical properties of a thermoset.

The elastic reinforcing strands 64 may also contain blends of elastic and inelastic polymers, or of two or more elastic polymers, provided that the blend exhibits elastic properties. The strands 64 are substantially continuous in length. The strands 64 may have a circular cross-section but, as previously mentioned, may alternatively have other cross-sectional geometries such as elliptical, rectangular, triangular or multi-lobal. In one embodiment, one or more of the elastic reinforcing strands 64 may be in the form of elongated, rectangular strips produced from a film extrusion die having a plurality of slotted openings.

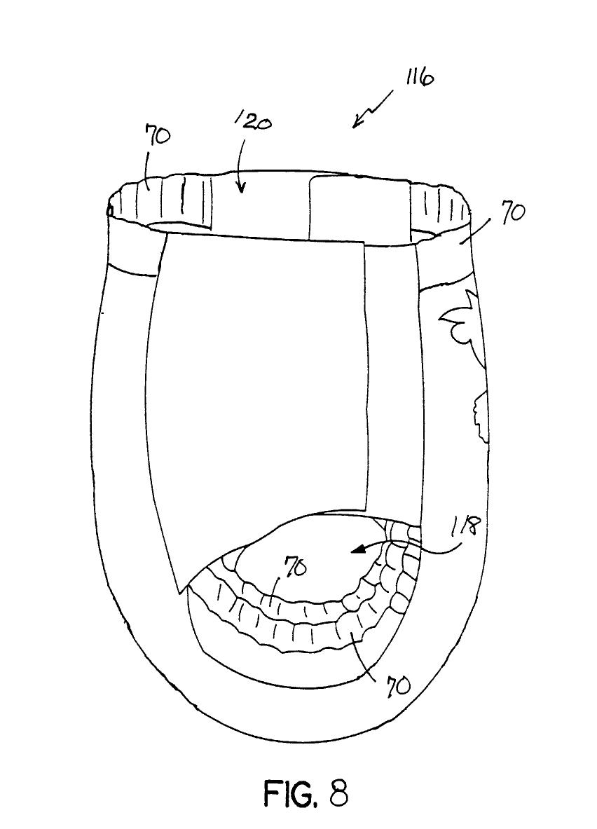

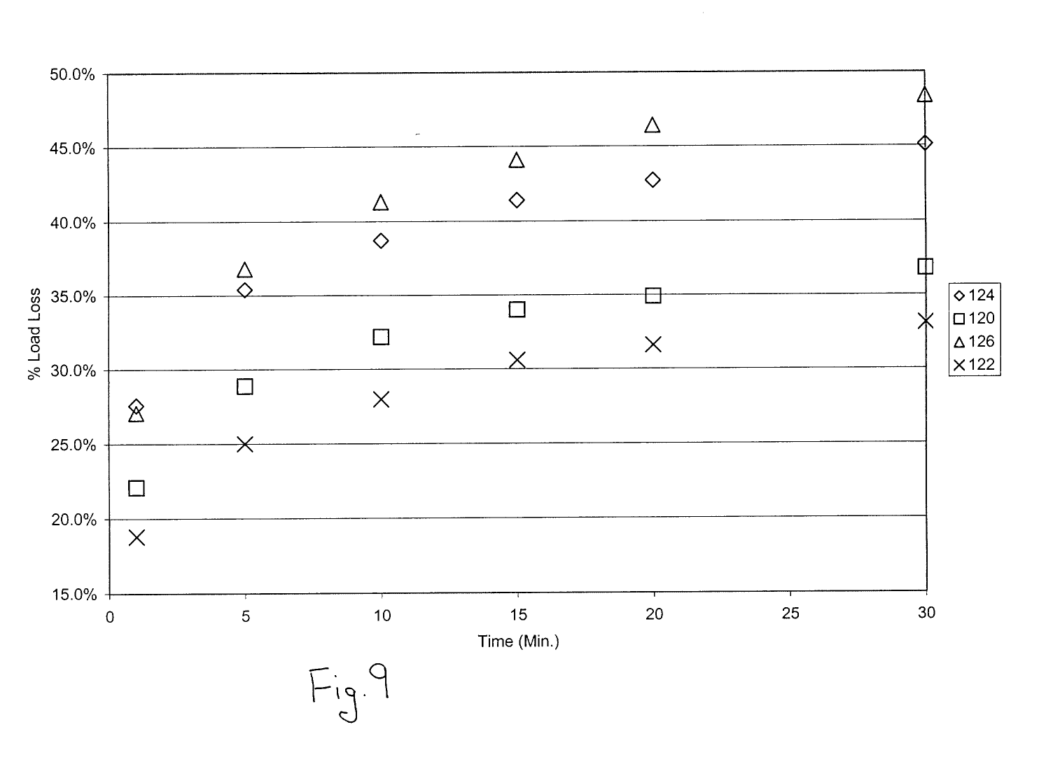

The elastic composite laminates 70 of the invention include the above-described elastic composites 60 sandwiched between a first facing sheet 72 and a second facing sheet 74, as shown in FIGS. 5 and 6. Facing materials may be formed using conventional processes, including the spunbond and meltblowing processes such as described above. For example, the facing sheets 72, 74 may each include a spunbond web having a basis weight of about 0.1-4.0 ounces per square yard (osy), suitably 0.2-2.0 osy, or about 0.4-0.6 osy. The facing sheets 72, 74 may include the same or similar materials or different materials. Alternatively, it is envisioned that laminates according to the present invention may be produced utilizing the stranded elastic barrier adhesive film placed between primary garment layers such as the back sheet 30 and topsheet 32 (FIG. 1).

If the facing sheets 72, 74 are to be applied to the composite 60, the facing sheets may be extensible or non-extensible depending upon the application. In one aspect of the invention, the facing sheets could be necked, or gathered, in order to allow them to be stretched after application of the elastic composite. Various post treatments, such as treatment with grooved rolls, which alter the mechanical properties of the material, may also be suitable for use.

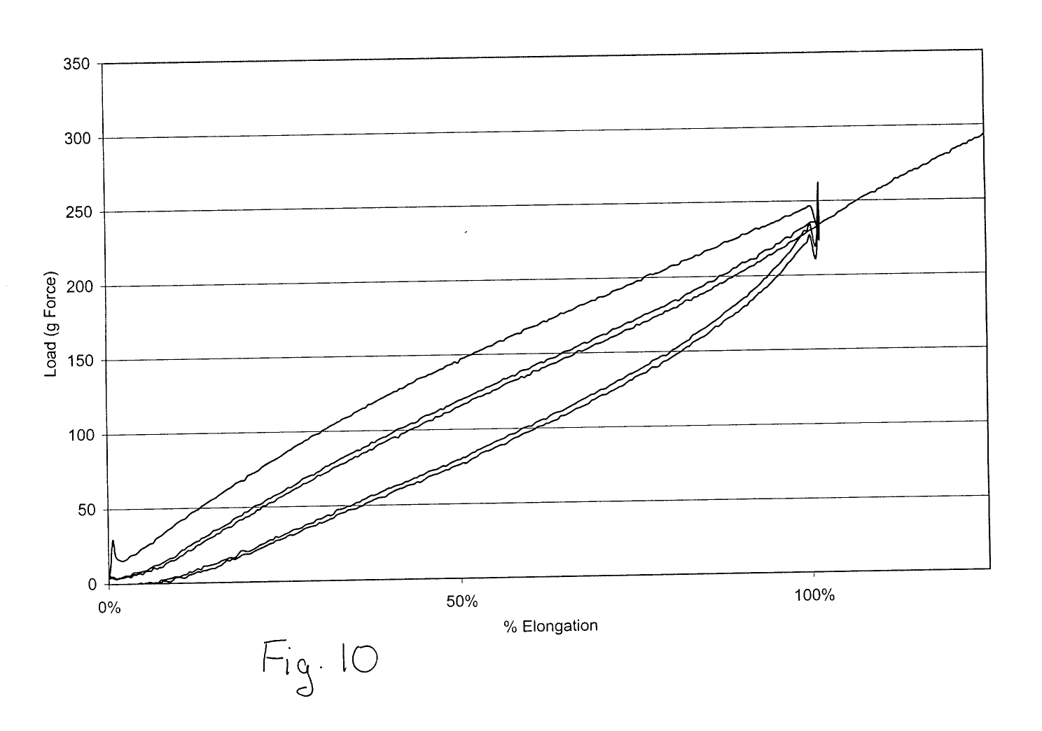

FIG. 7 illustrates a method and apparatus for making an elastic composite laminate 70 of the invention. While FIG. 7 illustrates a composite VF SBL process, it will be appreciated that other processes consistent with the present invention may be used. A first roller 76 provides reinforcing strands of elastic material 78 through a filament separator/guide 80 at, e.g. six strands per inch. The strands 78 are fed to a first tensioning roller 82 and stretched while conveyed vertically towards a nip 84 by one or more first fly rollers 86 in the strand-providing line to reach a desired prestretch of, e.g., 275%, at the nip. If desired, tension among the strands may be adjusted such that some strands have greater tension than others. For example, some strands may be stretched between about 150% and about 175% while other strands may be more desirably stretched between about 250% and about 275%. As another example of creating non-uniform tension among the elastic strands, some elastic strands may be incorporated into the elastomeric adhesive film at an add-on rate of between about 5 and about 50 grams per minute before stretching.

An extruder 88 using a slotted film die 90 produces the elastomeric adhesive film 92, such as made from Findley HX2695-F01 which is fed onto a chill roller 94 at about 120 gsm basis weight, and conveyed to one or more second fly rollers 96 towards the nip 84. The film 92 may be stretched down to a narrower width and thinned by the second fly rollers 96 during its passage to the nip 84 such as by a 700% (or 8×) stretch from chill roll to the nip. The nip 84 is formed by opposing first and second nip rollers 98, 100. The elastic composite 60 (not shown in this Fig.) is formed by incorporating the strands 78 into the elastomeric adhesive film 92 in the nip 84.

In order to form the elastic composite laminate 70, first and second rolls 106 and 108, respectively, of spunbond facing material, such as 0.5 osy spunbond nonwoven having fiber denier of approximately 2.0-2.5, containing approximately 50% Polyethylene and 50% Polyproplyene in a side-by-side configuration, and being thermally point bonded, are fed into the nip 84 on either side of the elastic composite and are bonded by the adhesive present in the elastic composite. The spunbond facing material might also be made in situ rather than unrolled from previously-made rolls of material. While illustrated as having two lightweight gatherable spunbond facings, it will be appreciated that only one facing material, or various types of facing materials, may be used. The elastic composite laminate 70 can be maintained in a stretched condition by a pair of tensioning rollers 110, 112 downstream of the nip 84 and then relaxed as at reference number 114.

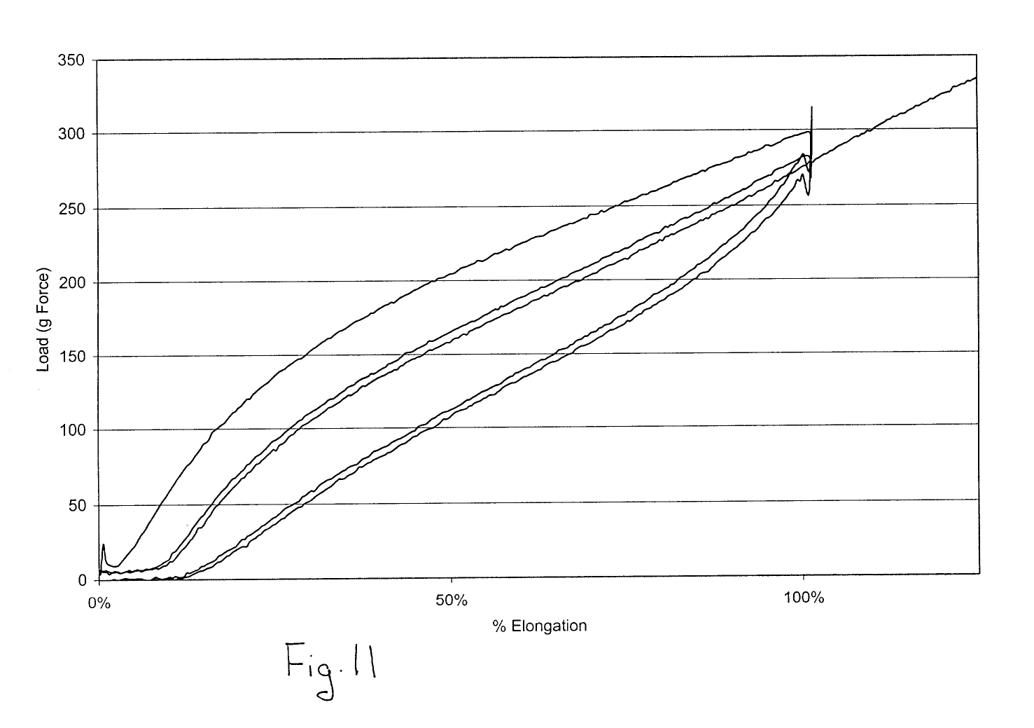

The resulting elastic composites and elastic composite laminates are particularly useful in providing elasticity in personal care absorbent garments 116, as shown in FIG. 8. More specifically, as shown in FIG. 8, the elastic composite laminates 70 are particularly suitable for use in providing a gasket-like fit around cuff areas such as leg openings 118 and waist openings 120. The laminates of this invention are less likely to undergo tension decay or delamination compared to similar laminates lacking the reinforcing strands, as demonstrated in the example below. Furthermore, the reinforcing strands enable the composite tension to be tunable while preserving the soft feel and aesthetic properties of the laminate. Thus, elastic composite laminates can be produced with a desired fit or gasket-like quality without causing red marks on a wearer's skin due to excessive tension, while preserving the soft and gentle feel and improved adhesion of the laminate.

### Test Methods

Adhesive Bond Strength

The adhesive bond strength of the elastomeric adhesive film of the present invention is determined as follows. A test sample of the elastic composite laminate having dimensions of about 2.0 inches (5.08 cm) wide by about 4.0 inches (10.16 cm) long, or as large as possible up to this size, is used for testing. The adhesive bond strength is determined through the use of a tensile tester, such as a SINTECH tensile tester commercially available from the Sintech Co., Cary, N.C., Model No. II. A 90 degree peel adhesion test is run in order to determine the grams of force needed to pull apart the first and second layers of facing sheet of the laminate. Specifically, 1.25 inches (3.175 cm) or more of the 4-inch length of the test sample has the first and second layers of facing sheet peeled apart. The first facing sheet is then clamped in the upper jaw of the tensile tester, and the second facing sheet is clamped in the lower jaw of the tensile tester. The tensile tester is set to the following conditions:

Crosshead speed: 300 millimeters per minute

Full-scale load: 5,000 grams

Start measurements: 10 millimeters

Gauge length (jaw spacings): 1.0 inch (2.54 cm)

The tensile tester is then engaged. The test is terminated after approximately 100 millimeters on a 2-inch by 2-inch sample. Twenty data points per second are collected for a total of about 400 data points. The average of these data points is reported as the adhesive bond strength. The results from the tensile tester are normalized to a sample having a width of 1 inch. At least three test samples are subjected to the above testing with the results being averaged and normalized to produce the reported adhesive bond strength.

Stress-Strain Cycle Test (2 Cycles to 100% Elongation as Presented in Table 1)

An elastic composite sample of 2 in. wide and 6 in. long is placed in the clamps of a constant rate of extension (CRE) load frame, such as the previously described SINTECH tensile tester. Starting at a 4 in. gauge length between the sample grips, the sample is elongated at 20 in./min. to 100% elongation (8 in. jaw-span). The cross-head returns to the original 4″ gauge length position and then repeats the previously described 100% cycle again for a second time. The sample is then elongated again for a third time to its ultimate elongation at which the sample yields and possibly breaks. The data points at 50% elongation on the first cycle (50% E1) and at 50% retraction on the second cycle (50% R2) are then taken to calculate the load loss at 50% over the 2 cycle test. Calculation of % Load Loss @ 50%=(50%E1−50% R2)/50%E2*100. Lower % loss values correspond to better elastic efficiency of the composite measured.

Stress-Relaxation Test (50% Elongation for 30 min. or 4 Hr. as Presented in Table 2)

An elastic composite sample of 2 in. wide and 6 in. long is placed in the clamps of a constant rate of extension (CRE) load frame, such as the previously described SINTECH tensile tester. Starting at a 4 in. gauge length between the sample grips, the sample is elongated at 20 in./min. to 50% elongation (6 in. jaw-span). The cross-head is held at the 50% elongation position (6 in. jaw-span) for the designated test duration (30 min. or 4 hr.). Load of force in g-force is recorded for the entire test. The percentage Load Loss is calculated from the Initial load at 50% at time equals zero minutes and the load at the end of test period. For example: Calculation of % Load Loss (30 min.)=[Load @50%(t=0 min.)−Load @50%(t=30 min.)]/(Load @ 50%(t=0 min.)*100.

Laminate Growth Test (10″ Original; Calculated % Growth After 130° F. Aging, as Presented in Table 3)

A laminate sample is prepared by measuring and cutting a fully stabilized piece of laminate to ten inches in length by two inches wide. A plastic ruler is used to press the laminate flat against a smooth work bench surface and to mark sample length with a black pen. The measurement should be made in the machine direction or in the direction of stretch. The laminate is cut straight across in the cross-machine direction. The sample is loosely placed without compression or physical hindrance into a plastic bag and then placed on a tray in a gravity convection oven such as Model OV-490-A3, available from BLUE M Electric Company, Homewood, Ill. The sample is allowed to remain in the oven for the designated period of time and then removed. The sample is allowed to condition at The Technical Association of the Paper and Pulp Industry (TAPPI) standard laboratory conditions for 4 hours. The sample is then measured in the same manner as described above.

### EXAMPLES

A first sample of laminate material was made with Findley HX2695-F01 as the EBA film and incorporating six spandex fibers, of 310 deciTex (dTex), per two inches of film with one quarter inch spacing between strands. The spandex strands were pre-tensioned to 275% elongation prior to pressure lamination. A second sample of laminate material was made with Findley HX2695-F01 as the EBA film and incorporating groups of 3 spandex fibers, of 310 deciTex (dTex), and 3 spandex fibers, of 210 deciTex (dTex), per two inches of film with one quarter inch spacing between strands. The spandex strands were pre-tensioned to 275% elongation prior to pressure lamination. Each sample was made according to the process described with respect to FIG. 7. A third, control sample was prepared without spandex fibers. The EBA film in each sample had 120 gsm basis weight at a 700% stretch before bonding to the nonwoven facings. Nonwoven facings in each sample were 0.5 osy spunbond nonwoven having fiber denier of approximately 2.0-2.5, containing approximately 50% Polyethylene and 50% Polyproplyene in a side-by-side configuration, having a thermally point bonded structure and being utilized on both sides of the composite. The samples were aged at room temperature and at 130° F. for 55 days for the first and control sample batches and for 30 days for the second sample batch, as reported in Table 1.

TABLE 1
STRESS-STRAIN CYCLE TEST
(2 CYCLES TO 100% ELONGATION) % Loss Fields are Calculated

| | | | | 2^{nd} cycle | | |
|---|---|---|---|---|---|---|
| | | | 1^{st} Elongation | retraction | | % Loss |
| Code | Storage | Time/Age | Tension @ 50% | tension @ 50% | % Set,Cycle 2 | @ 50% |
| control | RT | Day 55 | 157.7 | 70 | 14.9 | 55.6% |
| sample 1 | RT | Day 55 | 206.4 | 108.1 | 13.7 | 47.6% |
| control | 130° F. | Day 55 | 97 | 37.4 | 23.5 | 61.4% |
| sample 1 | 130° F. | Day 55 | 150.5 | 79.3 | 10.2 | 47.3% |
| sample 2 | RT | Day 30 | 219.3 | 116.9 | 8.3 | 46.7% |
| sample 2 | 130° F. | Day 30 | 159.6 | 83.7 | 8.7 | 47.6% |

TABLE 2
STRESS-RELAXATION TEST
(50% ELONGATION FOR 30 MIN. & 4 HR.)

| | | | Test Conditions | | |
|---|---|---|---|---|---|
| | | | TAPPI Std. | | 100° F./ |
| | | | (70° F./50% RH) | | 50% RH |
| | | | Stress- | Stress- | Stress- |
| | | | Relaxation | Relaxation | Relaxation |
| | | | % Load | % Load | % Load |
| | | | Loss | Loss | Loss |
| Code | Storage | Time/Age | 30 min. | 4 Hr. | 30 min. |
| control | RT | Day 55 | 43.9% | 49.5% | 45.1% |
| sample 1 | RT | Day 55 | 36.8% | 41.7% | 36.8% |
| control | 130° F. | Day 55 | 46.7% | 55.8% | 48.4% |
| sample 1 | 130° F. | Day 55 | 32.9% | 40.2% | 33.1% |

TABLE 3
% GROWTH REDUCTIONS/IMPROVEMENTS % DIMENSIONAL
GROWTH IN MACHINE DIRECTION AT 130° F.

| | | | | Laminate | Laminate | Laminate |
|---|---|---|---|---|---|---|
| | | | | Growth | Growth | Growth |
| Code | Storage | Time/Age | Lycra | 1 Day | 2 Day | 7 Day |
| control | 130° F. | 1,2,& 7 Day | None | 12.5% | 15.0% | 23.8% |
| sample 2 | 130° F. | 1,2,& 7 Day | 3 × 210 dTex, 3 × 310 | 2.5% | 5.0% | 5.0% |

As seen in Table 1, the percentage of tension loss in the two cycle stress-strain test was less for the samples prepared in accordance with the present invention than for the controls. As seen in Table 2, the percentage of stress relaxation was less for the samples prepared in accordance with the present invention than for the controls. As seen in Table 3, the percentage of laminate growth was less for the samples prepared in accordance with the present invention than for the controls.

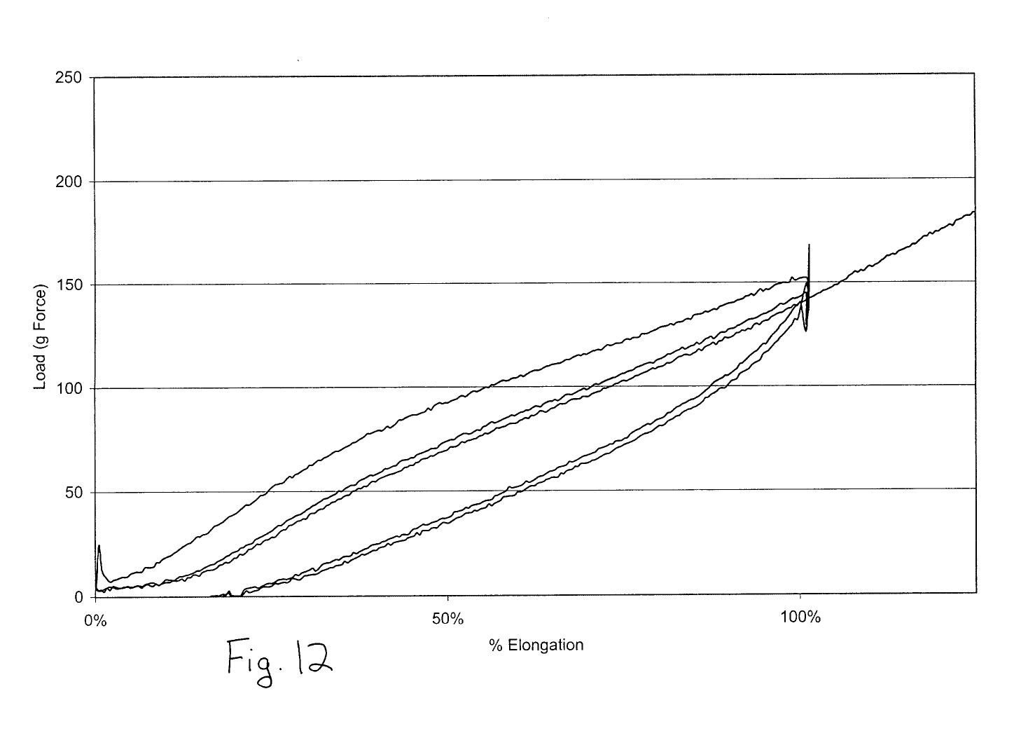

Further with respect to the elastic reinforced film/laminates, as seen in FIG. 9, a graph of the results of the Stress Relaxation Test, shows the percentage of Load Loss on the Y-axis versus the Time at 50% stretch on the X-axis, as taken at 100° F. and 50% relative humidity, for the first and control samples. Fifty percent stretch is taken as a realistic assumption of the strain on an actual diaper leg cuff opening at the time of donning. It can be seen that the laminates having spandex fibers added thereto, as indicated by data point series 120, 122, both aged 55 days and at room temperature and 130° F., respectively, exhibit less percentage loss of load over time than the controls, as indicated by data point series 124, 126, both aged 55 days and at room temperature and 130° F., respectively.

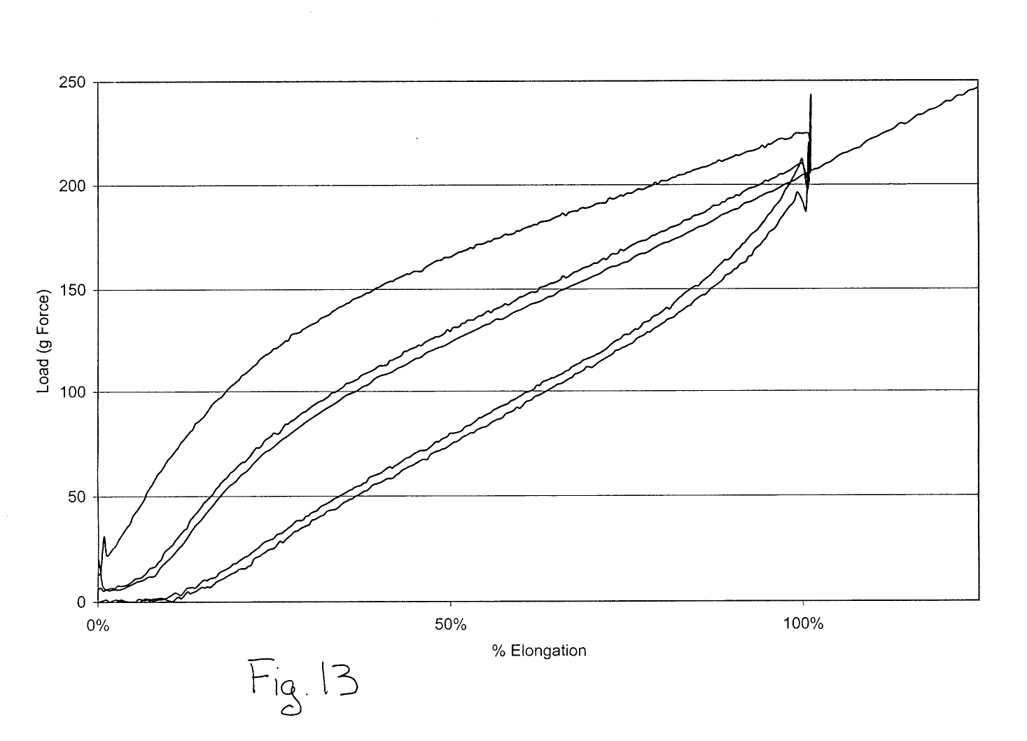

Further, with respect to the spandex-containing laminates, FIGS. 10-11 and 12-13 show graphs of the results of a Stress-Strain Cycle Test, for Sample 1 and the control sample, respectively, showing the percentage of Load Force on the Y-axis versus the Percentage of Elongation on the X-axis. A 100% cycle test stretching and relaxing the first and control samples from zero to 100% to zero elongation is indicated to show the hysteresis of the material. It can be seen that the laminates having spandex fibers added thereto, i.e., in FIGS. 10-11, both aged 55 days and at 130° F. and room temperature, respectively, exhibit greater load force retention, or greater elastic efficiency, over their range of elongation than the controls, i.e., in FIGS. 12-13, both aged 55 days and at 130° F. and room temperature, respectively.

From the foregoing examples it can be seen that better elongation tension, better second cycle retraction tension, and better hysteresis/elastic efficiency are obtainable by reinforcing the elastic barrier adhesive material with elastic, e.g., spandex, strands when making an expandable laminate for a garment or absorbent article.

It will be appreciated that details of the foregoing embodiments, given for purposes of illustration, are not to be construed as limiting the scope of this invention. Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention.

We claim:

1. A garment having at least one elastomeric composite therein, the elastomeric composite comprising a plurality of elastic strands incorporated into an elastomeric adhesive film; and
the garment having at least one expandable area utilizing the elastomeric composite.

2. The garment of claim 1, wherein the plurality of elastic strands comprises spandex.

3. The garment of claim 1, wherein the elastomeric adhesive film comprises an elastomeric, hot melt, pressure-sensitive adhesive.

4. The garment of claim 1, further comprising a first facing sheet bonded to a first surface of the elastomeric adhesive film.

5. The garment of claim 4, further comprising a second facing sheet bonded to a second surface of the elastomeric adhesive film.

6. The garment of claim 5, wherein at least one of the first and second facing sheets comprises a spunbond web.

7. The garment of claim 1, further comprising:
the elastomeric composite having a first set of the elastic strands having a first tension along a first portion of the film and a second set of the elastic strands having a second tension along a second portion of the film, the first tension greater than the second tension.

8. The garment of claim 7, wherein the first set of elastic strands has a different thickness than the second set of elastic strands.

9. The garment of claim 7, wherein the elastic strands in the first set of elastic strands are spaced more closely than the elastic strands in the second set of elastic strands.

10. The garment according to claim 1 wherein the expandable area is a waist area.

11. The garment according to claim 1 wherein the expandable area is a leg area.

12. The garment according to claim 1 wherein the expandable area is a containment flap.

13. The garment according to claim 1 wherein the expandable area is a side panel.

14. The garment according to claim 1 wherein the expandable area is a cuff area.

15. The garment according to claim 14 wherein the cuff area is a waistband.

16. The garment according to claim 14 wherein the cuff area is a leg opening.

17. The garment of claim 1, wherein the garment is one selected from the group of personal care garments, medical garments, and industrial workwear garments.

18. The garment of claim 1, wherein the garment is one selected from the group of: diapers, training pants, swim wear, absorbent underpants, adult incontinence products, feminine hygiene products, protective medical gowns, surgical medical gowns, caps, gloves, drapes, face masks, laboratory coats, and coveralls.

19. The garment of claim 2, wherein the elastomeric adhesive film comprises an elastomeric, hot melt, pressure-sensitive adhesive.

20. The garment of claim 19, further comprising a first facing sheet bonded to a first surface of the elastomeric adhesive film.

21. The garment of claim 20, further comprising a second facing sheet bonded to a second surface of the elastomeric adhesive film.

22. The garment of claim 21, wherein at least one of the first and second facing sheets comprises a spunbond web.

23. The garment of claim 22, wherein the garment is one selected from the group of personal care garments, medical garments, and industrial workwear garments.

24. The garment of claim 22, wherein the garment is one selected from the group of: diapers, training pants, swim wear, absorbent underpants, adult incontinence products, feminine hygiene products, protective medical gowns, surgical medical gowns, caps, gloves, drapes, face masks, laboratory coats, and coveralls.

25. The garment of claim 19, further comprising:
the elastomeric composite having a first set of the elastic strands having a first tension along a first portion of the film and a second set of the elastic strands having a second tension along a second portion of the film, the first tension greater than the second tension.

26. The garment of claim 25, wherein the first set of elastic strands has a different thickness than the second set of elastic strands.

27. The garment of claim 25, wherein the elastic strands in the first set of elastic strands are spaced apart more closely than the elastic strands in the second set of elastic strands.
